(19) United States
(12) Patent Application Publication
(10) Pub. No.: US 2008/0242731 A1
(43) Pub. Date: Oct. 2, 2008

(54) TOPICAL ANESTHESIA FORMULATION FOR BODILY CAVITIES

(75) Inventors: Thierry Vancaillie, Castlecrag (AU); Alan Hewitt, Bondi Junction (AU)

Correspondence Address: SEED INTELLECTUAL PROPERTY LAW GROUP PLLC, 701 FIFTH AVE, SUITE 5400, SEATTLE, WA, 98104, US

(73) Assignee: T&A PHARMA PTY. LIMITED

(21) Appl. No.: 12/055,172

(22) Filed: Mar. 25, 2008

Related U.S. Application Data
(63) Continuation-in-part of application No. 11/005,618, filed on Dec. 6, 2004.
Application No. 11/005,618 is a continuation-in-part of application No. 10/625,816, filed on Jul. 22, 2003, now abandoned.
(60) Provisional application No. 60/602,501, filed on Aug. 17, 2004.

(51) Int. Cl. A61K 31/164; A61K 47/04; A61P 17/02

(52) U.S. Cl. 514/626; 514/769

(57) ABSTRACT

The present invention relates to topical anaesthetic compositions, uses thereof and methods for their preparation.

### CROSS-REFERENCE TO RELATED APPLICATION

This application is a continuation-in-part of U.S. patent application Ser. No. 11/005,618, filed Dec. 6, 2004, now pending, which application is a continuation-in-part of U.S. Patent Application No. 10/625, filed Jul. 22, 2003, now abandoned and claims the benefit under 35 U.S.C. § 119(e) of U.S. Provisional Patent Application No. 60/602,501, filed Aug. 17, 2004, now expired; which applications are incorporated herein by reference in their entireties.

### FIELD OF THE INVENTION

The present invention relates generally to topical anaesthetic compositions, and more specifically to a combination of topical anaesthetic ingredients having pH, viscosity, and bioavailability properties suitable for use in reducing pain associated with a wound, trauma or other source of pain.

### BACKGROUND OF THE INVENTION

In their “natural” state, topical anaesthetics such as Lidocaine (also known as Lignocaine) have a low pH of 3.5 to 4.5, good solubility in aqueous solution, and potentially indefinite stability. Objectionably though, low pH anesthetics when applied to patients can be irritating and are not as active as when the pH is within the range of that of human tissues.

In contrast, it has been observed that an anaesthetic solution buffered to a pH of around neutral (pH=7) is less irritating and hence more acceptable to patients. The onset of action of buffered anaesthetics may be more rapid and more effective on prior inflamed tissue, however raising the pH from 3.5 to neutral makes the anaesthetic gradually less soluble and more likely to precipitate. When precipitated, the anaesthetic molecules are no longer bioavailable and exert no anaesthetic effect.

To compensate for the decrease in solubility caused by increasing pH, commercially available preparations of higher pH (i.e. neutral) anaesthetics limit their concentrations to a low percentage (<1%). This trade-off of concentration against pH maintains the anaesthetic in solution, however the active ingredients are subject to degradation in higher pH solutions. For example, lignocaine maintained at a pH of 7 even at low concentration can degrade 35% within 4 weeks. A low concentration of an anaesthetic with tissue-adjusted pH may be satisfactory for injection into the site of action, but not when the anaesthetic molecules must penetrate through the mucosa to reach the site of action. Whilst topical application offers numerous advantages in terms of ease of administration and patient comfort, topically applied anaesthetic compositions require high concentrations to promote penetration of the molecules. Low concentration preparations, even at a pH close to the target tissue, are not effective. Resorting to administering drugs intravenously causes major undesirable side effects.

There is therefore a need for topically applied compositions which deliver anaesthetic molecules at body-adjusted pH, whilst at the same time maintaining bioavailability.

### SUMMARY OF THE INVENTION

In a first aspect, the present invention provides a method of preparing a buffered anaesthetic composition, the method comprising the steps of: providing a buffering composition having a pH of between about 6.5 and about 11 which, when mixed with an anaesthetic agent having a pH in solution of between about 2.5 and about 5.0, yields a buffered anaesthetic composition having a pH of at least about 5.5; providing an unbuffered composition comprising an anaesthetic agent having a pH in the range of between about 2.5 and about 5.0, and at least 2% concentration; and mixing the buffering composition and the unbuffered composition so as to produce a buffered anaesthetic composition having a pH of at least 5.5.

The buffered anaesthetic composition may be a topical composition.

The pH of the buffered anaesthetic composition may be between about 5.5 and about 7.5, or between about 6.0 and about 7.0, or between about 6.5 and about 7.0, or between about 6.5 and about 7.5, or about 6.8.

The concentration of the anaesthetic agent in the unbuffered composition may be between about 2% and about 20%, or between about 2% and about 15%, or between about 2% and about 12%, or between about 2% and about 10%, or between about 2% and about 5%, or between about 5% and about 20%, or between about 5% and about 15%, or between about 7% and about 12%, or about 10%.

The concentration of the anaesthetic agent in the buffered anaesthetic composition may be between about 1% and about 20%, or between about 1% and about 15%, or between about 1% and about 12%, or between about 1% and about 10%, or between about 1% and about 5%, or between about 4% and about 5%, or about 1%, 2%, 3%, 4%, 5%, 6%, 7%, 8% or 9%.

The pH of the buffering composition may be between about 6.5 and about 11.0, or between about 6.5 and about 9.5, or between about 6.5 and about 9.0, or between about 6.5 and about 8.0, or between about 7.0 and about 8.0, or between about 7.5 and about 8.2, or about 7.8.

The method of the first aspect may further comprise the step of adding a solubilising agent to the buffering composition.

The solubilising agent may be selected from the group consisting of: propylene glycol, glycerol, ethanol, isopropanol, butylenediol, polyethylene glycol 100 to polyethylene glycol 600, N-methyl-2-pyrrolidone, dimethyl isosorbide, cyclodextrin and derivatives thereof, vitamin E polyethylene glycol succinate, diethylene glycol monoethyl ether, polyglyceryl oleate, polyglyceryl monocaprylate, polyglyceryl monolaurate, glyceryl monooleate, lecithin, polysorbates and combinations thereof.

The method of the first aspect may further comprise the step of adding a viscosity agent to at least one of the buffering composition or the unbuffered composition so as to increase the viscosity of either composition.

The viscosity agent may be selected from the group consisting of: cellulose and derivatives thereof, polyethylene glycol, alginates, branched polysaccharides, fumed silica, xanthan gum and polyacrylates. The viscosity agent may be Methocel™.

The method of the first aspect may further comprise adding a viscosity agent to both the buffering composition and the unbuffered composition so as to increase the viscosity of both compositions.

The viscosity agent may be added in about equal amounts to both the buffering composition and the unbuffered composition.

The amount of viscosity agent added to one or both of the buffering composition and the unbuffered composition may be between about 1% (w/w) and about 60% (w/w), or between about 1% (w/w) and about 50% (w/w), or between about 1% (w/w) and about 45% (w/w), or between about 2% (w/w) and about 40% (w/w) of the total mass of the buffering composition or the unbuffered composition.

The buffering composition may comprise bicarbonate.

The buffering composition may comprise phosphate.

The buffering composition may comprise dihydrogen phosphate (H₂PO₄⁻).

The buffering composition may comprise hydrogen phosphate (HPO₄²⁻).

The buffering composition may comprise dihydrogen phosphate and hydrogen phosphate.

The unbuffered composition and/or the buffering composition may further comprise one or more preservatives and/or antibacterial agents, for example benzyl alcohol or parabens.

The anaesthetic agent may be lidocaine, prilocaine, etidocaine, articaine, marcaine, carbocaine, bupivacaine, mepivacaine or any combination thereof.

In a second aspect, the present invention provides a method of preparing a topical buffered anaesthetic composition, the method comprising the steps of: providing a buffering composition comprising hydrogen phosphate, dihydrogen phosphate, N-methyl-2-pyrrolidone and hydroxypropyl methylcellulose having a pH in the range of between about 6.5 and about 8.5; providing an unbuffered composition comprising lignocaine and hydroxypropyl methylcellulose having a pH in the range of between about 2.5 and 5.0, and at least 2% concentration of lignocaine; and mixing the buffering composition and the unbuffered composition so as to produce a buffered anaesthetic composition having a pH of at least 5.5.

The buffering composition and the unbuffered composition may further comprise one or more preservatives, for example benzyl alcohol and/or parabens.

In a third aspect, the present invention provides a buffered anaesthetic composition whenever prepared by the process of the first or second aspects.

In a fourth aspect, the present invention provides a topical buffered anaesthetic composition comprising an anaesthetic agent, a viscosity agent and a solubilising agent, said composition having a pH between about 5.5 and 7.5.

The composition may have a pH between about 6.0 and about 7.0, or a pH of about 7.0, or about 6.8.

The viscosity agent and the solubilising agent may be as defined in the first aspect.

The composition may comprise the following components: a mixed phosphate buffer, hydroxypropyl methylcellulose and N-methyl-2-pyrrolidone.

In a fifth aspect, the present invention provides a topical buffered anaesthetic composition comprising lignocaine, N-methyl-2-pyrrolidone, hydroxypropyl methylcellulose, hydrogen phosphate and dihydrogen phosphate, said composition having a pH between about 5.5 and 7.5.

The composition of the fifth aspect may consist essentially of lignocaine, N-methyl-2-pyrrolidone, hydroxypropyl methylcellulose, hydrogen phosphate and dihydrogen phosphate.

In a sixth aspect, the present invention provides a buffered anaesthetic composition comprising an anaesthetic agent, a viscosity agent and a solubilising agent having a pH between about 5.5 and 7.5, when used as a topical anaesthetic composition.

In a seventh aspect, the present invention provides a kit comprising: a first component including a buffering composition having a pH such that when mixed with an anaesthetic solution having a pH of between about 2.5 and about 5.0, yields a buffered anaesthetic composition having a pH of between about 6.0 and about 7.5; a solubilising agent; and a viscosity agent; and a second component including a composition of an anaesthetic agent having a pH between about 3.0 and 5.0 and at least 2% concentration; and a viscosity agent.

The concentration of the anaesthetic agent may be between about 2% and about 20%, or between about 2% and about 15%, or between about 2% and about 12%, or between about 2% and about 10%, or between about 2% and about 5%.

The pH of the buffering composition may be between about 6.5 and about 11.0, or between about 6.5 and about 9.5, or between about 6.5 and about 9.0, or between about 6.5 and about 8.0, or between about 7.0 and about 8.0, or about 8.0.

The buffering composition may comprise dihydrogen phosphate and/or hydrogen phosphate.

The solubilising agent may be selected from the group consisting of: propylene glycol, glycerol, ethanol, isopropanol, butylenediol, polyethylene glycol 100 to polyethylene glycol 600, N-methyl-2-pyrrolidone, dimethyl isosorbide, cyclodextrin and derivatives thereof, vitamin E polyethylene glycol succinate, diethylene glycol monoethyl ether, polyglyceryl oleate, polyglyceryl monocaprylate, polyglyceryl monolaurate, glyceryl monooleate, lecithin, polysorbates and combinations thereof. In one embodiment, the solubilising agent may be N-methyl-2-pyrrolidone.

The viscosity agent may be selected from the group consisting of: cellulose and derivatives thereof, polyethylene glycol, alginates, branched polysaccharides, fumed silica and xanthan gum.

In an eighth aspect, the present invention provides a kit comprising: a first component including a buffering composition comprising hydrogen phosphate, dihydrogen phosphate, hydroxypropyl methylcellulose and N-methyl-2-pyrrolidone having a pH between about 6.5 and about 8.5; and a second component including an unbuffered composition comprising lignocaine and hydroxypropyl methylcellulose having a pH between about 3.0 and 5.0 and at least 2% concentration of lidocaine.

In a ninth aspect, the present invention provides a method for controlling pain associated with a wound, said method comprising applying to the wound, or regional area surrounding the wound, an effective amount of a buffered anaesthetic composition as defined in the third, fourth or fifth aspects for a period of time sufficient to induce analgesia.

In a tenth aspect, the present invention provides a method for controlling pain associated with a wound, said method comprising applying to the wound, or regional area surrounding the wound, an effective amount of a buffered anaesthetic composition comprising lignocaine, hydroxypropyl methylcellulose, N-methyl-2-pyrrolidone, hydrogen phosphate and dihydrogen phosphate.

The wound may be the result of surgery.

The surgery may be a hysteroscopic procedure, a laparoscopic procedure, a cystoscopic procedure, an arthroscopic procedure, an endoscopic procedure, or any other type of open procedure.

The composition may be applied before, during or after surgery.

The wound may be located within a body cavity.

The body cavity may be selected from the group consisting of: the anus, the vagina, the uterine tract including the cervix, the uterine cavity, ostia or tubal mucosa. In one embodiment, the body cavity may comprise the region extending from the outside rim of the cervix to the fimbriated end of the fallopian tube.

In an eleventh aspect, the present invention provides a method of delivering a near neutral pH topical anaesthetic agent to a wound, or regional area surrounding the wound, to cause a rapid onset anaesthetic effect without precipitation of the anaesthetic agent.

The mixture may comprise a viscosity agent and/or a solubilising agent as defined above.

In a twelfth aspect, the present invention provides a method of delivering a near neutral pH topical anaesthetic composition comprising lignocaine, hydroxypropyl methylcellulose, N-methyl-2-pyrrolidone, hydrogen phosphate and dihydrogen phosphate to a wound, or regional area surrounding the wound, to cause a rapid onset anaesthetic effect without precipitation of the lignocaine.

Definitions

In the context of this specification, the terms “a” and “an” are used herein to refer to one or to more than one (i.e. to at least one) of the grammatical object of the article. By way of example, “an element” means one element or more than one element.

In the context of this specification, the term “comprising” means “including principally, but not necessarily solely”. Furthermore, variations of the word “comprising”, such as “comprise” and “comprises”, have correspondingly varied meanings.

In the context of this specification, the terms “anaesthetic agent” or “anaesthetic” are understood to include local and regional anaesthetics. Local anaesthetics may be ester or amide based, and include pharmaceutically acceptable salts, derivatives and prodrugs thereof.

In the context of this specification, the term “near neutral” as it relates to pH is understood to mean a pH of 7.0±0.5.

In the context of this specification, the term “body cavity” is understood to mean a permanent body cavity. The body cavity may be a body cavity in direct communication with the outside of the body, such as the vagina, anus, mouth or uterus, or the body cavity may be a body cavity not in direct communication with the outside of the body, such as the abdominal, pelvic, thoracic or cranial cavity.

In the context of this specification, the term “viscosity agent” is understood to be a compound or compounds that increase the viscosity of the composition to which they are added.

In the context of this specification, the term “solubilising agent” is understood to mean a compound or compounds which serve to prevent precipitation of the anaesthetic.

In the context of this specification, the term “rapid onset anaesthetic effect” is understood to mean the onset of an anaesthetic effect commencing within at least 10 minutes of anaesthetic administration.

### DETAILED DESCRIPTION OF THE INVENTION

An unbuffered solution of an amide anaesthetic (i.e. at acidic pH) can remain stable for years, however once it is buffered the solution will remain stable for only a few days before precipitation occurs, thereby significantly lowering bioavailability. In order to address this problem of insolubility following buffering, the present invention provides, in one aspect, two separate compositions (a buffering composition and an unbuffered composition), which may be combined prior to use. As such, the present invention stores the anaesthetic component and the buffer component separately, which allows maximisation of the buffer pH independently of the anaesthetic concentration. The present invention also encompasses buffered anaesthetic compositions prepared by addition of the separate compositions, and their use in methods of performing topical anaesthesia. Once prepared, the buffered anaesthetic compositions of the invention are stable for several hours, and in terms of efficacy, ease of use, application and persistence, they are highly effective.

Mixing a high pH buffer solution with a high (>2%) concentration anaesthetic solution produces a high (>1%) concentration aqueous solution of topical anaesthetic having a pH of about 7. This combination may verge on instability such that a small increase in pH may cause the anaesthetic agent to become insoluble and to precipitate, thereby dramatically reducing the bioavailability of the anaesthetic. Therefore, in one embodiment of the invention a mixed phosphate base (e.g. dihydrogen phosphate and hydrogen phosphate) buffering solution having a pH of between about 6.5 and about 8 may be used as the buffering composition, so as to maximise the margin of solubility. However, alternative bases such as bicarbonate or hydroxide, or indeed any other suitable buffering components known to those skilled in the art may also be used.

The anaesthetic agent used in the unbuffered composition may be an amide anaesthetic (for example lidocaine) at a concentration of >2%, wherein the pH of the solution is between about 3.5 and 4.5, or about 3.8. Other known amide anaesthetics such as prilocaine, etidocaine, articaine, bupivacaine and mepivacaine, or combinations thereof, may also be used. Alternatively, ester anaesthetics such as procaine, tetracaine or chloroprocaine may be used.

TABLE 1
Ranges of pH and concentration in separate components and mixture

| | Unbuffered | Buffering | Buffered |
|---|---|---|---|
| | Solution | Solution | Solution |
| pH | 2.5-5 | 6.5-11 | 5.5-7.5 |
| Conc. of anaesthetic | >2% | 0 | >1% |

As shown in Table 1, the pH range of the unbuffered composition may be between about 2.5 and about 5.0, or between about 2.5 and about 4.5, or between about 2.5 and about 4.25, or between about 3.0 and about 4.0, or between about 3.5 and about 4.5, or about 3.8.

The pH range of the buffering composition may be between about 6.5 and about 11.0, or between about 6.5 and 10.5, or between about 6.5 and 10.0, or between about 6.5 and about 9.5, or between about 6.5 and about 9.0, or between about 6.5 and about 8.0, or between about 7.0 and about 8.0, or between about 7.3 and about 8.2, or about 6.5, 6.6, 6.7, 6.8, 6.9, 7.0, 7.1, 7.2, 7.3, 7.4, 7.5, 7.6, 7.7, 7.8, 7.9 or 8.0.

The pH range of the buffered anaesthetic composition may be between about 5.5 and about 7.5, or between about 6.0 and about 7.0, or between about 6.5 and about 7.0, or between about 6.5 and about 7.5, or about 6.8, or about 6.0, 6.1, 6.2, 6.3, 6.4, 6.5, 6.6, 6.7, 6.8, 6.9 or 7. The pH of the buffered anaesthetic composition may be near neutral.

In solution, molecules of anaesthetic may combine, crystallize and precipitate as agglomerates which are no longer able to penetrate through tissue and exert their effect. In order to minimise this possibility, the buffering composition or the unbuffered composition may further include a solubilising agent. The solubilising agent inhibits crystallization of the anaesthetic molecules, thereby maximising solubility. This enables the composition to hold a higher concentration of the anaesthetic agent and thereby increases the bioavailability, potency and effect of the anaesthetic agent. Upon mixing the buffering and unbuffered compositions, any small quantities of anaesthetic agent that begin to crystallize are immediately solubilised and prevented from acting as seed sites for precipitation.

The solubilising agent may be selected from the group consisting of: propylene glycol, glycerol, ethanol, isopropanol, butylenediol, polyethylene glycol 100 to polyethylene glycol 600, N-methyl-2-pyrrolidone, dimethyl isosorbide, cyclodextrin and derivatives thereof, vitamin E polyethylene glycol succinate, diethylene glycol monoethyl ether, polyglyceryl oleate, polyglyceryl monocaprylate, polyglyceryl monolaurate, glyceryl monooleate, lecithin, polysorbates and combinations thereof The solubilising agent may be added to the buffering composition prior to mixing, or to the unbuffered composition prior to mixing, or to both compositions prior to mixing. The solubilising agent may be present in an amount of between about 2% (w/w) and about 30% (w/w), or between about 2% (w/w) and about 20% (w/w) or between about 2% (w/w) and about 15% (w/w), or between about 2% (w/w) and about 10% (w/w) of the buffering composition or the unbuffered composition.

In order to obtain a suitable viscosity, a viscosity agent may be added to either the buffering composition or the unbuffered composition. The viscosity agent may be added in an amount such that the buffered anaesthetic composition has sufficient viscosity to enable adherence to a surface of, and surrounding a wound or trauma. The viscosity agent may be a gel or gel-like carrier or other viscous agent, for example a polymer matrix. Viscosity agents are known to those skilled in the art. Examples of suitable viscosity agents may include cellulose and derivatives thereof (for example hydroxyethyl cellulose, hydroxypropyl cellulose, methyl cellulose, ethyl cellulose, hydroxyethylmethyl cellulose, hydroxyethylethyl cellulose, and hydroxypropylmethyl cellulose), guar gum, carbomer, polyethylene glycol, alginates, branched polysaccharides, fumed silica and xanthan gum.

The amount of viscosity agent added to one or both of the buffering composition and the unbuffered composition may be between about 1% (w/w) and about 60% (w/w), or between about 1% (w/w) and about 50% (w/w), or between about 1% (w/w) and about 45% (w/w), or between about 2% (w/w) and about 40% (w/w), or between about 2% (w/w) and about 35% (w/w) of the total mass of either the buffering composition or the unbuffered composition. In one embodiment, the amount of viscosity agent added to one or both of the buffering composition and the unbuffered composition may be between about 2% (w/w) and about 10% (w/w), or between about 25% (w/w) and about 40% (w/w) of the total mass of either the buffering composition or the unbuffered composition.

Topical anaesthetics are typically applied directly to their site of action. This may be relatively simple when the target tissue is visible to the physician and accessible without the need to pass body openings. However, when the target tissue is within a body cavity, tissue debris, exudates, secretions and blood may provide a barrier. In order to displace this material, the viscosity of the buffered anaesthetic composition may be higher than that of blood, and high enough to assist the composition in adhering to a portion of the skin of the body cavity. An advantage in having a viscosity slightly higher than that of blood is that capillary force can attract the composition comprising the anaesthetic molecules to remain within the bodily cavity for the time required for the anaesthetic molecules to transfer from the liquid vehicle into the target tissue.

The viscosity should however not be significantly higher than that of blood so as to preserve the composition's ability to flow easily and enter intended-to-be-anesthetized regions of a bodily cavity that are inaccessible to more viscous gels. Accordingly, the viscosity agent may be provided in such an amount so as to displace blood and exudates, whilst at the same time not preventing the product from reaching all areas of the body cavity. The viscosity of the buffered anaesthetic composition may be between about 4 cps and about 450 cps, or between about 4 cps and about 400 cps, or between about 5 cps and about 375 cps, or between about 5 cps and 350 cps, or between about 4 cps and about 150 cps, or between about 5 cps and about 100 cps, or between about 4 cps and about 50 cps, or between about 300 cps and about 400 cps, or between about 315 cps and about 390 cps, or between about 325 cps and 375 cps, or between about 340 cps and 360 cps.

In one embodiment, the viscosity agent may be added in substantially the same amount to both the buffering composition and the unbuffered composition. The viscosity agent may be hydroxypropyl methylcellulose (for example Methocel™, available as 2% Methocel™ syrup). The viscosity agent effectively maintains the viscosity of the constituent composition, and is generally unaffected by pH in the range of 3 to 11. Sonography has shown that a buffered anaesthetic composition in accordance with the present invention having a level of viscosity between about 6 cps and 350 cps, is able to penetrate and be retained in uterine cavities for several hours after instillation.

Permeation enhancers may also be added to the buffered composition if desired to improve absorption of the anaesthetic agent. Suitable permeation enhancers include, but are not limited to: sulfoxides, for example DMSO, azones, for example laurocapram, alcohols, alkanols, glycols, terpenes, sodium lauryl sulfate, EDTA, sodium cholate and 5-methoxysalicylate.

In an embodiment of the invention, the unbuffered composition may comprise lignocaine and a viscosity agent (for example hydroxypropyl methylcellulose) and the buffering composition may comprise a mixed phosphate buffer and a solubilising agent (for example N-methyl-2-pyrrolidone), and optionally a viscosity agent. In an alternative embodiment, the unbuffered composition may comprise lignocaine, a viscosity agent (for example hydroxypropyl methylcellulose) and a preservative, and the buffering composition may comprise a mixed phosphate buffer, a solubilising agent (for example N-methyl-2-pyrrolidone), and a viscosity agent. The viscosity agent may be present in about the same amount in both compositions.

In another embodiment of the invention the unbuffered composition may comprise an anaesthetic agent in an amount of between about 5% and about 15% of the total weight of the unbuffered composition, and a viscosity agent in an amount of between about 2% and about 12% of the total weight of the unbuffered composition, and the buffering composition may comprise a phosphate buffer, a viscosity agent in an amount between about 2% and about 12% of the total weight of the buffering composition, and a solubilising agent in an amount between about 3% and about 15% of the total weight of the buffering composition. Preservatives and/or antibacterial agents may also be added to one or both compositions. Sterile water may also be added to both the buffering composition and the unbuffered composition. In this embodiment, the viscosity agent may optionally be omitted from the buffering composition. The unbuffered composition may have a pH of about 4.2, the buffering composition may have a pH of about 7.8, and the pH following mixing of the buffering composition and the unbuffered composition may be about 6.8.

In a further embodiment of the invention the unbuffered composition may comprise an anaesthetic agent in an amount of between about 4% and about 10% of the total weight of the unbuffered composition, and a viscosity agent in an amount of between about 20% and about 40% of the total weight of the unbuffered composition, and the buffering composition may comprise a phosphate buffer, a viscosity agent in an amount between about 20% and about 40% of the total weight of the buffering composition, and a solubilising agent in an amount between about 2% and about 15% of the total weight of the buffering composition. Preservatives and/or antibacterial agents may also be added to one or both compositions. Sterile water may also be added to both the buffering composition and the unbuffered composition. The unbuffered composition may have a pH of about 4.2, the buffering composition may have a pH of about 7.8, and the pH following mixing of the buffering composition and the unbuffered composition may be about 6.8.

Mixing of the buffering and unbuffered compositions may be performed just prior to use, or alternatively a few hours prior to use. Combining the unbuffered and buffering compositions in a 1:1 ratio by weight produces a >1% anaesthetic concentration aqueous composition having a pH of about 7. In order to delay coalescence and postpone precipitation of the anaesthetic agent within the mixture of the two compositions, the two compositions may be rapidly mixed together in a technique known as “speed mixing”. This technique typically involves drawing one of the components (usually the buffering composition) into a syringe, fitting a needle, and then emptying the syringe into the other component as rapidly as possible. The composition is then applied to a wound, or regional area surrounding a wound, or a position where trauma has occurred, which results in excellent bioavailability and effectiveness of the constituent anaesthetic.

The buffered compositions may be applied to any wound, trauma or other source of pain. The viscosity of the compositions may be such that they are able to adhere to the surface of a wound and effectively deliver the anaesthetic agent so as to prevent pain. Application of the compositions to a wound may be carried out by any means including spraying, painting, direct application by finger or swab or impregnation of a dressing. The buffered compositions may be used for the prevention of pain associated with wounds made during surgical procedures and may be applied before, during or after the surgical procedure. The buffered compositions are also advantageous in that they provide effective anaesthesia in the case of surgical wounds that are located within body cavities.

The buffered composition is administered in a therapeutically effective amount. A therapeutically effective amount will vary depending on the nature of the wound and the type and condition of the subject, however the amount is an amount which inhibits the experience of pain, lessens existing pain, prevents pain from worsening or eliminates pain altogether.

The buffered compositions may be adapted for use in both human and non-human animals, and hence have human and veterinary applications.

### EXAMPLES

### Example 1

### Compositions

The following compositions were prepared in accordance with the invention:

Composition 1

| Unbuffered composition: | | |
|---|---|---|
| Lignocaine HCl monohydrate | 23.7 | g |
| Benzyl alcohol | 200 | μL |
| 2% Methocel ™ syrup | 10 | g |
| Sterile water | 200 | mL |
| Buffering composition (pH 7.8): | | |
| Benzyl alcohol | 200 | μL |
| Dipotassium phosphate | 6.0 | g |
| Potassium dihydrogen phosphate | 2.0 | g |
| Pharmasolve | 20 | mL |
| 2% Methocel ™ syrup | 10 | g |
| Sterile water | 200 | mL |

Composition 2

| | | |
|---|---|---|
| Unbuffered composition: | | |
| Lignocaine HCl monohydrate | 11.85 | g |
| Benzyl alcohol | 100 | μL |
| 2% Methocel ™ syrup | 50 | g |
| Sterile water | 100 | mL |
| Buffering composition (pH 7.8): | | |
| Benzyl alcohol | 100 | μL |
| Dipotassium phosphate | 3.0 | g |
| Potassium dihydrogen phosphate | 1.0 | g |
| Pharmasolve | 10 | mL |
| 2% Methocel ™ syrup | 50 | g |
| Sterile water | 100 | mL |

Composition 3

| | | |
|---|---|---|
| Unbuffered composition: | | |
| Lignocaine HCl monohydrate | 23.7 | g |
| Methyl parabens | 200 | mg |
| 2% Methocel ™ A4M aqueous syrup | 20 | g |
| Sterile water | 200 | mL |
| Buffering composition (pH 7.8): | | |
| Methyl parabens | 200 | mg |
| Dipotassium phosphate | 6.0 | g |
| Potassium dihydrogen phosphate | 2.0 | g |
| N-methyl-2-pyrrolidone | 20 | mL |
| Sterile water | 200 | mL |

### Example 2

### Hysteroscopic Sterilization Study

In this study, two cohorts of patients were compared. The first cohort of patients underwent a hysteroscopic procedure without a buffered anaesthetic composition. 22% of these patients needed to be converted to inhalation anaesthesia as a result of pain. The second cohort of 17 patients underwent the procedure with topical buffered composition 1 from Example 1 above, applied as follows. The buffered anaesthetic composition was prepared just prior to application. 8 cc was then drawn into a syringe and the syringe attached to a catheter. The catheter was then introduced into the uterine cavity and about 3 cc of buffered anaesthetic was instilled therein. None of the second cohort of patients needed any other form of anaesthesia for the procedure.

### Example 3

### Cervical Compliance Study

A cervical compliance study was performed which compared two groups of patients. The first group received buffered gel without anaesthetic, and the second group received buffered composition 1 from Example 1 above applied in the same manner described above in Example 2. Both groups contained 20 subjects. The difference in force needed to pass the cervix was measured (in N/cm²). A reduction in force needed is considered an increase in compliance. In the groups receiving buffered composition 1, cervical compliance improved by 0.37 N. The clinical significance of this is that it is easier to pass instruments and minimize pain due to the reduced force required to overcome surgical resistance.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of preparing a buffered anaesthetic composition, the method comprising the steps of: providing a buffering composition having a pH of between about 6.5 and about 11 which, when mixed with an anaesthetic agent having a pH in solution of between about 2.5 and about 5.0, yields a buffered anaesthetic composition having a pH of at least about 5.5; providing an unbuffered composition comprising an anaesthetic agent having a pH in the range of between about 2.5 and about 5.0, and at least 2% concentration; and mixing the buffering composition and the unbuffered composition so as to produce a buffered anaesthetic composition having a pH of at least 5.5.

2. The method of claim 1, wherein the pH of the buffered anaesthetic composition is between about 5.5 and about 7.5.

3. The method of claim 2, wherein the pH of the buffered anaesthetic composition is between about 6.5 and 7.5.

4. The method of claim 1, further comprising the step of adding a solubilising agent to the buffering composition.

5. The method of claim 1, further comprising the step of adding a viscosity agent to at least one of the buffering composition or the unbuffered composition so as to increase the viscosity of either composition.

6. The method of claim 5, further comprising adding a solubilising agent to the buffering composition.

7. The method of claim 5, further comprising adding a viscosity agent to both the buffering composition and the unbuffered composition so as to increase the viscosity of both compositions.

8. The method of claim 7, wherein the viscosity agent is added in about equal amounts to both the buffering composition and the unbuffered composition.

9. The method of claim 1, wherein the buffering composition comprises bicarbonate or phosphate.

10. The method of claim 1, wherein the anaesthetic agent is selected from the group consisting of: lidocaine, prilocaine, etidocaine, articaine, marcaine, carbocaine, bupivacaine, mepivacaine or any combination thereof.

11. A topical buffered anaesthetic composition comprising an anaesthetic agent, a viscosity agent and a solubilising agent, said composition having a pH between about 5.5 and 7.5.

12. The composition of claim 11, wherein the pH is between about 6.5 and about 7.2.

13. The composition of claim 11, wherein the viscosity agent is selected from the group consisting of: cellulose and derivatives thereof such as hydroxypropyl methylcellulose, polyethylene glycol, alginates, branched polysaccharides, fumed silica, xanthan gum and polyacrylates.

14. The composition of claim 11, wherein the solubilising agent is selected from the group consisting of: propylene glycol, glycerol, ethanol, isopropanol, butylenediol, polyethylene glycol 100 to polyethylene glycol 600, N-methyl-2-pyrrolidone, dimethyl isosorbide, cyclodextrin and derivatives thereof, vitamin E polyethylene glycol succinate, diethylene glycol monoethyl ether, polyglyceryl oleate, polyglyceryl monocaprylate, polyglyceryl monolaurate, glyceryl monooleate, lecithin, polysorbates and combinations thereof.

15. The composition of claim 13, wherein the viscosity agent is hydroxypropyl methylcellulose.

16. The composition of claim 14, wherein the solubilising agent is N-methyl-2-pyrrolidone.

17. A kit comprising: a first component including a buffering composition having a pH such that when mixed with an anaesthetic solution having a pH of between about 2.5 and about 5.0, yields a buffered anaesthetic composition having a pH of between about 6.0 and about 7.5; a solubilising agent; and a viscosity agent; and a second component including a composition of an anaesthetic agent having a pH between about 3.0 and 5.0 and at least 2% concentration; and a viscosity agent.

18. The kit of claim 17, wherein the pH of the buffering composition is between about 6.5 and about 9.5

19. The kit of claim 17, wherein the buffering composition comprises dihydrogen phosphate and/or hydrogen phosphate.

20. The kit of claim 17, wherein the solubilising agent is selected from the group consisting of: propylene glycol, glycerol, ethanol, isopropanol, butylenediol, polyethylene glycol 100 to polyethylene glycol 600, N-methyl-2-pyrrolidone, dimethyl isosorbide, cyclodextrin and derivatives thereof, vitamin L polyethylene glycol succinate, diethylene glycol monoethyl ether, polyglyceryl oleate, polyglyceryl monocaprylate, polyglyceryl monolaurate, glyceryl monooleate, lecithin, polysorbates and combinations thereof.

21. The kit of any one of claim 17, wherein the viscosity agent is selected from the group consisting of: cellulose and derivatives thereof, polyethylene glycol, alginates, branched polysaccharides, fumed silica, xanthan gum and polyacrylates.

22. A method for controlling pain associated with a wound, said method comprising applying to the wound, or regional area surrounding the wound, an effective amount of a buffered anaesthetic composition as defined in claim 11.

23. The method of claim 22, wherein the wound is a result of surgery.

24. The method of claim 22, wherein the composition is applied before, during or is 5 after the surgery.

25. The method of claim 22, wherein the wound is located within a body cavity.

26. The method of claim 25, wherein the body cavity is selected from the group consisting of: the anus, the vagina, the uterine tract including the cervix, the uterine cavity, ostia or tubal mucosa.
